(19) United States
(12) Patent Application Publication
(10) Pub. No.: US 2026/0137848 A1
(43) Pub. Date: May 21, 2026

(54) DIALYSIS MACHINE WITH A DETACHABLE, REVERSIBLE SUPPORT PLATE, SUPPORT PLATE, USE OF A SUPPORT PLATE IN A DIALYSIS MACHINE, AND SYSTEM COMPRISING A DIALYSIS MACHINE AND A FLUID CONTAINER

(71) Applicant: B. Braun Avitum AG, Melsungen (DE)

(72) Inventor: Rolf Bertram, Bergheim (DE)

(21) Appl. No.: 19/386,947

(22) Filed: Nov. 12, 2025

(30) Foreign Application Priority Data
Nov. 19, 2024 (DE) 10 2024 133 958.6

(51) Int. Cl. A61M 1/14

(52) U.S. Cl. CPC A61M 1/14; A61M 2205/15; A61M 2209/084

(57) ABSTRACT

A dialysis machine includes a removable support plate for a fluid container. The support plate has a first surface and a second opposing surface. The first surface has at least one projection. The second opposing surface differs from the first surface, and can be level or flat. The at least one projection is configured to engage in or be accommodated in a corresponding notch of the fluid container. The support plate is inserted into a receptacle section of the dialysis machine in a first insertion orientation and a second insertion orientation, depending on the shape of the fluid container. The first surface faces the receptacle section when the support plate is in the first insertion orientation. The second surface faces the receptacle section when the support plate is in the second insertion orientation.

### CROSS-REFERENCE TO RELATED APPLICATION

This application claims priority under 35 U.S.C. § 119 to German Application No. 10 2024 133 958.6, filed on Nov. 19, 2024, the content of which is incorporated by reference herein in their entirety.

### FIELD

The present invention relates to a dialysis machine with a support for fluid containers.

### BACKGROUND

Dialysis fluid or dialysis fluid concentrate for dialysis machines can be stored in fluid containers that are fastened to the dialysis machine or placed on or next to the dialysis machine. For this purpose, known dialysis machines have a base with a support or resting surface on which the fluid containers with dialysis fluid or dialysis fluid concentrate can be placed or stored. The dialysis machines can be mounted on casters and can therefore be moved between different treatment rooms. Since the fluid containers are attached to the dialysis machine, they can be conveniently moved together with the dialysis machine. However, when moving the dialysis machine, there is a risk that the fluid containers may tip over due to uneven floors or other vibrations. It is therefore common practice to secure the fluid containers to the dialysis machine using straps or buckles. However, securing the fluid containers is an additional step that must be performed by the user of the dialysis machine.

Fluid containers for dialysis fluid or dialysis fluid concentrate are known, for example, from DE 10 2022 118 053 A1.

It is also known that dialysis machines have holders that are adapted to the shape of at least certain fluid containers. This allows the fluid container to be easily inserted into the corresponding holder. An example of such a holder for a fluid container is known from EP 3 325 037 B1. This principle is similar to the well-known drink holders in cars. However, the holders usually only fit a specific container shape. This means that only fluid containers sold by the same manufacturer as the dialysis machine will usually fit into the holder. In practice, however, a wide variety of fluid containers in different shapes are available on the market and in use in hospitals.

### SUMMARY

The purpose of the present disclosure is therefore to overcome or at least reduce the disadvantages of the prior art and, in particular, to provide a dialysis machine to which fluid containers of different shapes or geometries can be easily and securely fastened.

According to the disclosure, this task is apparently solved by a dialysis machine according to claim 1. Furthermore, the task of the present disclosure is solved by a system according to claim 14. The task is also solved by using a support plate according to claim 16 and a support plate according to claim 17.

The present disclosure relates to an extracorporeal blood treatment machine or a dialysis machine with a removable/detachable support plate for at least one fluid container, in particular for a container for dialysis fluid or dialysis fluid concentrate. The support plate has a first surface and a second surface opposite the first surface. The first surface has at least one (convex) projection/dome or one (convex) bulge. The at least one (convex) projection is designed and formed to engage in or be accommodated in a corresponding (concave) notch/indentation of the fluid container. The second opposite surface differs from the first surface. The second opposite surface can, for example, be (essentially) level/planar/flat. Depending on the shape of the fluid container, the support plate is inserted into a base/receptacle section of the dialysis machine in a first insertion orientation or a second insertion orientation. In the first insertion orientation, the first surface faces toward the base/receptacle section, and in the second insertion orientation, the second surface faces toward the base/receptacle section.

In other words, the support plate is inserted into the receptacle section/base of the dialysis machine in such a way that it is dependent on the shape/outer contour of the fluid container, so that a side/surface of the support plate that matches the shape of the fluid container faces upwards or towards the fluid container. This means that the support plate can be inserted into the base/receptacle section of the dialysis machine in a first orientation and in a further orientation that is inverted relative to the first orientation.

In other words, the dialysis machine has a reversible (support) plate or a reversible plate on which the fluid container or containers can be placed or positioned. The turning plate can be aligned in the appropriate direction based on the shape of the fluid containers and can thus (together with the receptacle section/base) form a support surface/resting surface suitable for the fluid container.

The at least one (convex) projection may be a bulge that protrudes (essentially perpendicularly) from the first surface and thus functions as a fastening element. The (convex) projection can therefore protrude from the first surface in a dome shape. This means that the shape of the protruding dome-shaped projection allows a dome to be formed, which has the shape of a dome/convex curvature. The (convex) projection may in particular be round/rotationally symmetrical. The projection may be adapted in shape to the notch in the fluid container so that the projection together with the corresponding notch forms a fastening.

The support plate can essentially be designed as a flat plate with a first surface and a second surface opposite the first surface. A circumferential surface may be formed between the two surfaces. The support plate can, of course, have several, in particular two, projections. A separate fluid container can then be placed on each of the multiple projections.

The receptacle section/base can be designed as part of the dialysis machine housing. The receptacle section/base can form a support or resting surface for the fluid containers, which is accessible from above. The receptacle section/base can also protrude from the housing like a balcony.

The fluid container may in particular be a fluid container for dialysis fluid or dialysis fluid concentrate. The fluid in the fluid container may be an acidic or alkaline concentrate. Dialysis fluid can be made from water mixed with an acidic component and a basic component. The acidic component may be a solution containing sodium chloride, potassium chloride, magnesium chloride, calcium chloride, glucose, acetic acid, and/or citric acid, or acetate. The alkaline component can be sodium hydrogen carbonate or (bi)carbonate, for example. The fluid container may alternatively be a container for a disinfectant.

The fluid container may be in the form of a (round) bottle or a (square) canister. Both forms may have in common that the fluid container has a floor section from which a single circumferential side wall or several opposite side walls protrude. In particular, a (round) bottle may have a (round) floor section which has a support surface for contact with the support plate and a recessed section which forms the concave notch. The notch in the bottle is designed and shaped to accommodate the (convex) projection. The projection can engage in the notch in such a way that both components are fastened to each other. Preferably, an outer surface of the projection can bear against an inner surface of the notch so that a frictional connection can be achieved.

The canister/canister-shaped fluid container may have an essentially flat floor section. This means that an outer surface of the fluid container on the floor side can be essentially flat. This allows the canister to have a large support surface on the support plate.

The dialysis machine according to the disclosure allows fluid containers of different shapes or geometries to be placed on and/or fixed to the dialysis machine. Depending on the shape of the fluid container, a user may select the appropriate support plate geometry and insert the support plate into the dialysis machine such that the appropriate side/geometry faces up/toward the fluid container. If the fluid container is a bottle with a notch in the floor section, the user can insert the support plate into the receptacle section/base so that the second surface faces the receptacle section/base and thus toward the floor. This causes the opposite first surface to face upward or toward the fluid container, thereby forming a resting surface/shelf surface/support surface for the fluid container. However, due to the at least one (convex) projection, the resting surface does not form a smooth/flat/level surface, but rather a (resting) surface with one or more fastening elements. When the fluid container is then placed on the support plate, the (convex) projection of the support plate engages in the corresponding concave notch of the fluid container on the support plate, or is at least accommodated therein. This allows an outer surface of the projection (friction-locked) to bear against an inner surface of the notch. This interlocking mechanism can fasten the fluid container to the support plate and thus to the dialysis machine. However, it may also be provided, as disclosed, that the outer surface of the projection does not abut the inner surface of the notch. In this case, the projection of the support plate serves more as a positioning aid for the fluid container in order to place the fluid container in a suitable position, for example so that another fluid container can be placed on the support plate next to this fluid container.

The (convex) projection can also be or serve as a positioning aid in addition to or as an alternative to the fastening or fixing. The projection indicates to the user placing the fluid container on the support plate where the optimum position for the fluid container is. This ensures that another fluid container can be placed next to the first fluid container. Since the filled fluid containers can be heavy, it is advantageous if the fluid containers need to be lifted as little as possible by the user.

If a fluid container has the shape of a canister with a flat floor section, the user can insert the support plate into the receptacle section/base so that the first surface faces toward the receptacle section/base. The second surface therefore points in the opposite direction, upward/toward the fluid container. This means that the flat/level surface becomes a (substantially level) resting surface/shelf surface/support surface for the fluid container. This allows the canister to be placed on a flat/level resting surface with its flat floor section. The flat/level resting surface fits the flat floor section of the machine, ensuring that the canister stands securely on the support plate and does not tip over even when the dialysis machine is moved. The flat/level resting surface provides a universal receptacle that can accommodate fluid containers or canisters with different floor sections.

The task of the present disclosure is further solved by a system comprising the dialysis machine according to one of the above aspects and at least one fluid container.

Advantageous further developments of the disclosure are the subject of the appended subclaims.

According to an optional aspect of the present disclosure, the receptacle section/base may have a recess/indentation/receptacle/clearance into which the (convex) projection extends when the support plate is inserted in the first insertion orientation. This means that the projection can be accommodated in the recess. In other words, the receptacle section/base has a support section for the fluid containers and the (central) recess. The recess extends from the support section toward the floor/toward the wheels of the dialysis machine. Since the projection of the support plate can be immersed in the recess, the projection does not rest directly on a support section of the receptacle section/base and therefore does not protrude from the receptacle section/base.

Preferably, the recess can be adapted to the shape of the support plate. This means that the recess can be shaped in such a way that the support plate can be inserted at least partially into the recess.

Preferably, the receptacle section can have a circumferential groove surrounding the recess, with the support plate resting on an end of the circumferential groove. In other words, the recess may have a circumferential support surface on which the support plate can rest. The support surface can be formed as a step in a side wall of the recess. This means that the support surface can protrude from the side wall of the recess into the interior of the recess and can also be spaced parallel (in the direction of a floor of the recess) from the support section. The clearance between the support surface and the support section can correspond in particular to the thickness/height of the support plate. This allows the support plate to be flush with the support section and form an essentially flat support or resting surface for the fluid containers. The support surface can be designed such that the support plate and a floor of the recess are always spaced apart. This allows any liquid that has leaked from one of the fluid containers to always drain away.

The recess with the support surface on which the support plate rests can together form a frame for the support plate. This means that the support plate can be inserted into the frame and secured by the frame to prevent it from slipping sideways, i.e., in the width direction.

According to a further optional aspect of the present disclosure, a gap may be formed between the support surface or the frame and the support plate. This means that there may be a clearance between the support surface and the support plate. The gap can be formed both in the longitudinal or width direction of the support plate and in the height or thickness direction of the support plate. The gap in the longitudinal or transverse direction of the support plate can be designed such that the support plate is smaller in length and/or width than the frame or recess in which the support plate is enclosed. This can cause a gap to form between a circumferential surface of the support plate formed between the two opposing surfaces and the frame. The gap in the height or thickness direction of the support plate can be formed, for example, by spacers. Liquid that has leaked or been spilled from one of the fluid containers can drain away from the support plate through the gap. The liquid can flow into the recess or into the interior of the receptacle section/base, where the liquid is collected.

Preferably, the floor of the recess is inclined relative to the horizontal/forms an angle with the horizontal so that the liquid flows in a defined direction and collects at a predetermined location at the floor of the recess.

Preferably, the gap between the support plate and the support surface can be formed by spacers/support pins/distance pins that form a clearance between the support plate and the support surface. The spacers can protrude vertically from the first surface and/or the second surface of the support plate. The spacers can be individual pins spaced apart from each other. However, the spacers can also be designed as thin protruding webs interrupted by individual passages. The spacers may preferably be arranged in an edge section of the support plate. This allows the spacers to rest on the support surface. Furthermore, the spacers can rest against the side of the frame and prevent the support plate from slipping sideways.

Alternatively, the spacers/support pins/distance pins can also be formed on the support surface. The spacers can protrude from the support surface, particularly upwards or towards the support plate.

The support plate may preferably have openings for handling. The support plate may have an indentation extending from one surface to the opposite surface through the thickness of the support plate. This allows the user to reach into the support plate with their fingers and easily grasp the support plate, which sits flush in the frame.

According to a further optional aspect of the present disclosure, the at least one (convex) projection may form an opening on the second surface. The opening can be closed with a lid. Since the support plate has an approximately constant wall thickness, the (convex) projection on the first surface can form a corresponding concave opening. Liquid or bacteria can accumulate in the opening, making it difficult to clean the support plate. It may therefore be advantageous to close the opening with the lid. The lid allows the second surface to be essentially flat/level and to form a flat resting surface in the second insertion orientation.

The lid can be made of plastic and welded to the support plate. The lid can be fastened to the support plate, in particular by laser welding.

The opening can also be designed without a lid. The second surface may therefore have one or more deep indentations. Fluid can accumulate in the recesses, especially when the support plate is inserted in the second insertion orientation. If the openings are left open, material, and manufacturing costs for the lids or for applying the lids can be saved.

Preferably, the at least one projection may have a bore/fluid outlet bore, which is positioned in particular at a point of the projection that is furthest away from the first surface. Fluid can flow from the support plate into the recess through the fluid outlet bore.

According to a further optional aspect of the present disclosure, the support plate may be inclined relative to the horizontal. The support surface on which the support plate rests in the receptacle section/base may be inclined at an angle relative to the horizontal. The angle of inclination can be between 2° and 5°, preferably 3°. The angle allows liquid on the support plate to flow off in a defined direction. The fluid can thus collect in a predetermined location/recess/basin. The liquid can be wiped or sucked off from the predetermined collection basin. The support surface of the receptacle section/base can also be inclined in relation to the horizontal. This allows a continuous support surface for the fluid containers to be formed.

Preferably, the dialysis machine may have a leakage sensor attached to the receptacle section/base. The leakage sensor can detect when liquid accumulates in the receptacle section/base. The leakage sensor can be connected to a control unit that processes a message about detected liquid and sends it to the user. The leakage sensor can, for example, be located at the lowest point on the floor of the recess.

Alternatively, the support plate can feature a leakage sensor. The leakage sensor can detect when liquid accumulates on the support plate.

According to an optional aspect of the present disclosure, the support plate may only have a projection. This allows two adjacent spaces to be formed for different fluid containers. A resting space for a bottle could be formed in the floor section with a notch, and a storage space for a canister with a flat floor section could be formed next to it.

The support plate can also form a projection on each of the two surfaces.

The support plate can preferably be fixed to the receptacle section/base with one or more magnets. Alternatively, the support plate can also be fixed to the receptacle section/base by means of a form fit or another detachable connection.

The (convex) projection can preferably be detachably fastened to the support plate. For example, the projection may be clipped or screwed. The projection can also be fastened to the support plate using another detachable joining method.

The lid can be welded onto the support plate, in particular by laser welding. Alternatively, the lid can also be glued or fastened to the support plate using any other material-locking joining method.

The support plate can be manufactured by injection molding, for example. Alternatively, the support plate can be manufactured using thermoplastic foam molding (TSG), reaction injection molding (RIM), or deep drawing. The support plate can also be 3D printed. The support plate can also be manufactured using a twin-sheet method. Two semi-finished products can be deep-drawn and joined simultaneously, enabling double-walled components to be formed applying a simple method.

PE, PP, PET, or other weldable plastics, especially thermoplastics, can be used as materials for the support plate.

Furthermore, the task of the present disclosure is solved by the use of the support plate in or on the dialysis machine, in particular a dialysis machine as described above. In particular, the support plate can be used as a base/resting surface/shelf for the fluid container on the dialysis machine. The support plate has the first surface and the second opposite surface. The first surface has at least one projection. The at least one projection is designed and formed to engage in or be accommodated in the corresponding notch of the fluid container. The second opposite surface differs from the first surface. In particular, the second opposite surface may be flat or planar. Depending on the shape of a section of the floor of the fluid container, the support plate is inserted into the receptacle section of the dialysis machine in the first insertion orientation or the second insertion orientation, or can be inserted into these. In the first insertion orientation, the first surface faces toward the receptacle section, and in the second insertion orientation, the second surface faces toward the receptacle section.

The use of the support plate as described in the disclosure allows fluid containers of different shapes to be arranged/fastened to the dialysis machine or placed securely on (the receptacle section) of the dialysis machine.

Furthermore, the present disclosure relates to a support plate for a dialysis machine, in particular a dialysis machine as described above, with the first surface and the second opposing surface. The first surface has at least one projection, and the second opposite surface differs from the first surface. The at least one projection is designed and formed to engage in or be accommodated in the corresponding notch of the fluid container. The support plate can be designed and constructed to be inserted into a receptacle section of the dialysis machine in a first insertion orientation or a second insertion orientation, depending on the shape of a floor section of the fluid container. Depending on the direction of insertion, one of the first and second surfaces may face upward or toward the receptacle section of the dialysis machine.

The support plate, as disclosed, allows for quick and easy modification of the receptacle geometry on the dialysis machine.

Preferably, the at least one projection can form the opening on the second surface. Preferably, the support plate can have a lid that closes the opening. The lid may be welded to the opening.

Advantageously, the first surface and/or the second surface may further comprise protruding spacers that may protrude concavely from the respective surfaces.

The support plate can also be designed without the protruding spacers. This can be advantageous in that the surface of the support plate is then flat/level and can therefore accommodate the canister-shaped fluid container particularly well. In this case, the spacers can be advantageously formed on the frame of the receptacle section of the dialysis machine.

Especially if the support plate does not have spacers, it may be advantageous to provide a geometry that prevents the support plate from slipping. For example, the receptacle section may have one or more recesses into which one or more corresponding projections on the support plate can engage. Of course, the support plate can also have one or more recesses and the receptacle section can have one or more corresponding projections. The combination of recess and projection can then prevent the support plate from slipping unintentionally relative to the receptacle section.

It is of course also conceivable that the support plate only has spacers on one of the two surfaces.

The at least one projection of the support plate may furthermore have a bore, which may be positioned in particular at a point of the projection that is furthest away from the first surface. Fluid can flow out of the opening through this bore, especially if the opening is not closed by a lid.

### BRIEF DESCRIPTION OF THE DRAWINGS

FIG. 1 shows a dialysis machine according to the present disclosure;

FIG. 2 shows a support plate of a dialysis machine according to a first embodiment of the present disclosure in a first insertion orientation;

FIG. 3 shows a longitudinal section through a section of the dialysis machine according to the first embodiment of the present disclosure;

FIG. 4 shows the support plate of the dialysis machine according to the first embodiment of the present disclosure in a second insertion orientation;

FIG. 5 shows a section of the dialysis machine according to the first embodiment of the present disclosure without a support plate;

FIG. 6 shows a first surface of the support plate of the dialysis machine according to the first embodiment of the present disclosure;

FIG. 7 shows a second surface of the support plate of the dialysis machine according to the first embodiment of the present disclosure;

FIG. 8 shows a base of the dialysis machine according to the first embodiment of the present disclosure with two fluid containers on the support plate in the first insertion orientation;

FIG. 9 shows a base of the dialysis machine according to the first embodiment of the present disclosure with two fluid containers on the support plate in the second insertion orientation;

FIG. 10 shows a support plate of the dialysis machine according to the second embodiment of the present disclosure in the first insertion orientation;

FIG. 11 shows the support plate of the dialysis machine according to the second embodiment of the present disclosure in the second insertion orientation;

FIG. 12 shows a support plate of the dialysis machine according to a third embodiment of the present disclosure; and

FIG. 13 shows a support plate of the dialysis machine according to a fourth embodiment of the present disclosure.

### DETAILED DESCRIPTION

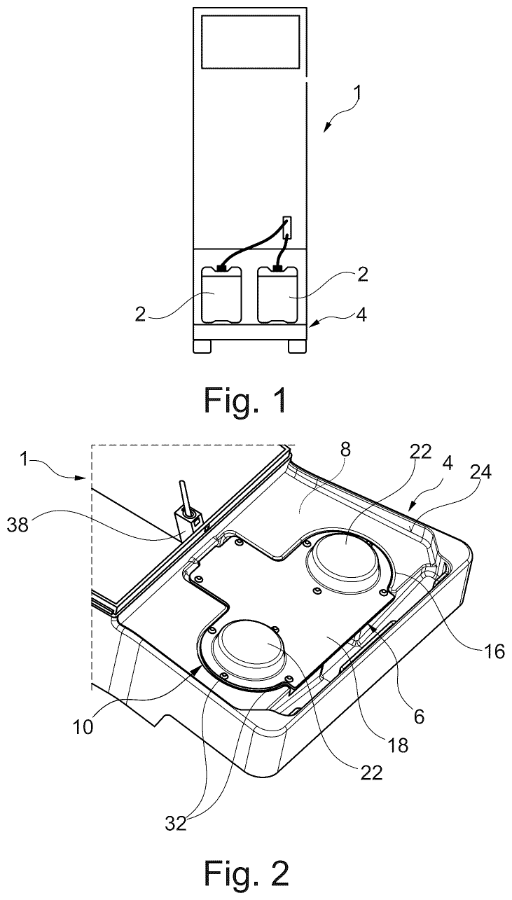

FIG. 1 shows a dialysis machine 1 with two fluid containers 2. The dialysis machine 1 has rollers on the bottom side of the dialysis machine 1 and can therefore be moved. The fluid containers 2 are mounted on a receptacle section/resting section/base 4 of the dialysis machine 1. This allows the fluid containers 2 to be moved together with the movable dialysis machine 1.

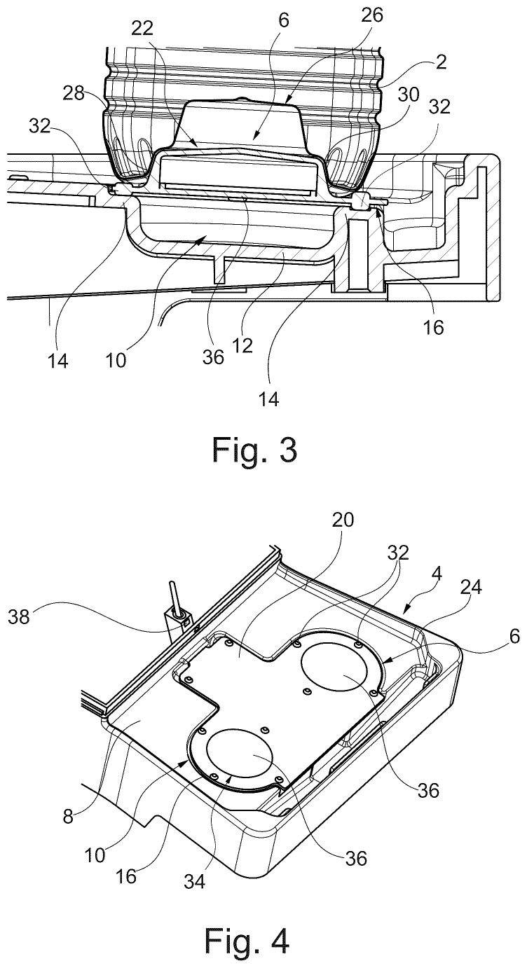

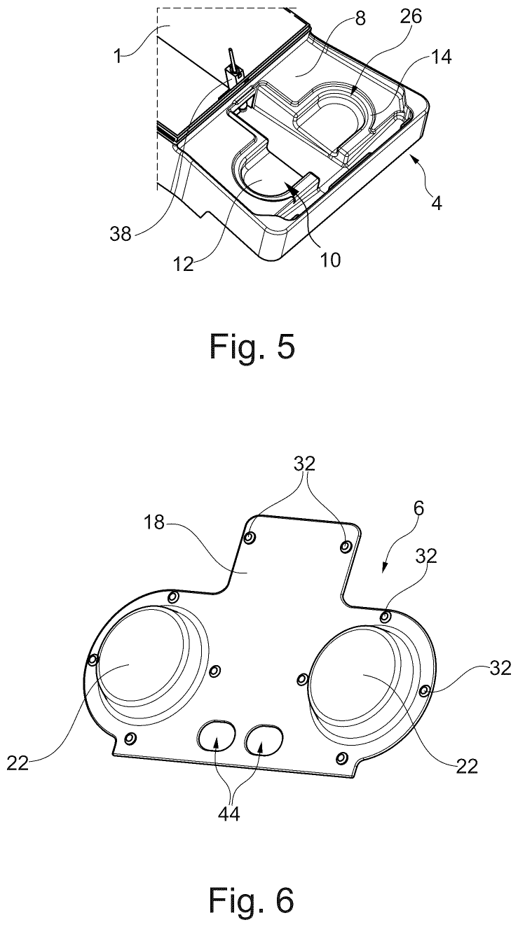

FIG. 2 shows a support plate 6 resting in a first insertion orientation on or in the base 4. The base 4 has an essentially flat support section 8 for the fluid containers 2 and a central frame/receptacle/clearance/recess 10 for the support plate 6. The recess 10 essentially has the shape of the support plate 6. This means that an inner contour of recess 10 is adapted to an outer contour of support plate 6. A floor 12 of the recess 10 (shown in FIG. 5) is offset downward/toward the floor of the dialysis machine 1 relative to the support section 8. Thus, recess 10 forms a clearance in base 4. The recess 10 has a bearing surface 14 (shown in FIG. 3) on which the support plate 6 rests. The support surface 14 is spaced parallel to the support section 8 and projects inward into the recess 10. The support surface 14 and the inner contour of the recess thus form a frame for the support plate 6. The clearance between the support surface 14 and the support section 8 essentially corresponds to the thickness of the support plate 6. The support plate 6 thus rests on the support surface 14 in such a way that the support plate 6 and the support section 8 are flush and form a common surface/plane. The support plate 6 thus forms, together with the support section 8, an essentially continuous shelf or resting surface for the fluid containers 2. The receptacle in the frame prevents lateral slipping of the support plate 6. The longitudinal or lateral extension of the support plate 6 is smaller than the frame/recess 10. This creates a gap 16 between the support plate 8 and the recess 10. Liquid can flow through the gap 16 from the support plate 8 into the recess 10 of the base 4 and collect in the recess 10.

The support plate 6 has a first surface 18 and a second opposite surface 20 (shown in FIG. 4). The first surface 18 has two convex projections 22 that protrude from the first surface 18. In the first insertion orientation, the support plate 6 is aligned so that the second surface 20 faces the base 4. Thus, the first surface 18 points upward away from the base 4 and forms a resting surface for the fluid containers 2. The support section 8 is offset parallel to a wall end/edge 24 of the base 4 so that the edge 24 protrudes beyond the support section 8. This allows fluid that leaks from the fluid containers 2 to be collected in the base 4.

When the fluid containers 2 are placed on the base 4, the projections 22 engage in corresponding notches 26 on a floor section of the respective fluid container 2. This allows the fluid container 2 to be fixed or secured in its position. This prevents the fluid container 2 from falling over when the dialysis machine 1 is moved. Furthermore, the projections 22 indicate the optimal position for the individual fluid containers 2. This ensures that two fluid containers 2 can be placed side by side on the base 4.

FIG. 3 shows a section through the base 4 with the support plate 6 in the first insertion orientation, with one of the fluid containers 2 standing on the support plate 6. The convex projection 22 engages in the notch 26 of the fluid container 2. The notch 26 and the projection 22 are designed such that an outer surface 28 of the projection 22 rests against an inner surface 30 of the notch 26. The projection 22 thus supports the fluid container 2 and therefore functions as a fastening element.

The support plate 6 has spacers/spacer pins/distance pins 32 that protrude essentially vertically from the second surface 20. When the spacers 32 rest on the support surface 14, the clearance/gap 16 between the support plate 6 and the support surface 14 is formed. Spilled liquid from the fluid containers 2 can run through this gap 16 into the recess 10 of the base. The spacers 32 also rest against the recess 10, thereby preventing the support plate 6 from slipping sideways.

FIG. 4 shows the support plate 6 in a second insertion orientation. In the second insertion orientation, the first surface 18 faces toward the base 4 and the second surface 20 faces upward, forming the resting surface. The second surface 20 is level/flat. This means that the level second surface 20 and the flush support section 8 of the base 4 together form an essentially flat/level resting surface or shelf surface. Since the support plate 6 has an essentially constant plate thickness, the convex projections 22 each form a corresponding (concave) opening/bulge 34 in the second surface 20. These openings 34 are each closed by lids 36 so that the second surface 20 is flat and no liquid or bacteria can collect in the opening. The flat resting or shelf surface formed in this way is designed so that canister-shaped fluid containers 2 with flat floors can be placed on it. The canisters have a large support surface on the support plate 6 and are therefore well secured against tipping over.

In summary, the support plate 6 can be inserted into the base 4 in the desired/appropriate insertion orientation based on the shape of the fluid containers 2. This ensures that the side/surface that best fits the shape of the fluid containers 2 always faces upwards.

FIG. 5 shows base 4 without support plate. The base has the support section 8 and the recess 10, which is formed centrally in the support section 8. The recess 10 is adapted to the shape of the support plate 6. The recess 10 has the support surface 14, which is spaced parallel to the support section 8. The support surface 14 and the contour of the recess form the frame for the support plate 8. A leakage sensor 38 is located at the lowest point of recess 10. The leakage sensor 38 detects liquid that collects in the recess and sends a warning to a control unit (not shown) of the dialysis machine 1. The floor 12 of the recess 10 is inclined relative to the horizontal so that liquid collects at a defined location. The floor 12 is inclined toward the dialysis machine 1. The support surface 14 is also inclined relative to the horizontal. Thus, the support plate 6, which rests on the support surface 14, also forms an angle with respect to the horizontal. This allows fluid to drain away from the support plate 6. The fluid flows preferentially through the gap 16 between the support plate 6 and the frame into the recess 10.

FIG. 6 shows the first surface 18 of the support plate 6. The support plate 6 essentially has an elliptical base shape 40 with a fastening section 42 that protrudes from the elliptical base shape. The two round convex projections 22 protrude from the first surface 18. The support plate 6 has handling openings 44 for handling the support plate 6. The support plate 6 further has a number of protruding spacers 32. The spacers 32 rest on the support surface 14 and thus form the gap 16 or a clearance between the support surface 14 and the support plate 6 when the support plate 6 is inserted into the base 4. Spilled liquid can flow through this gap 16 into the recess 10 in the base 4.

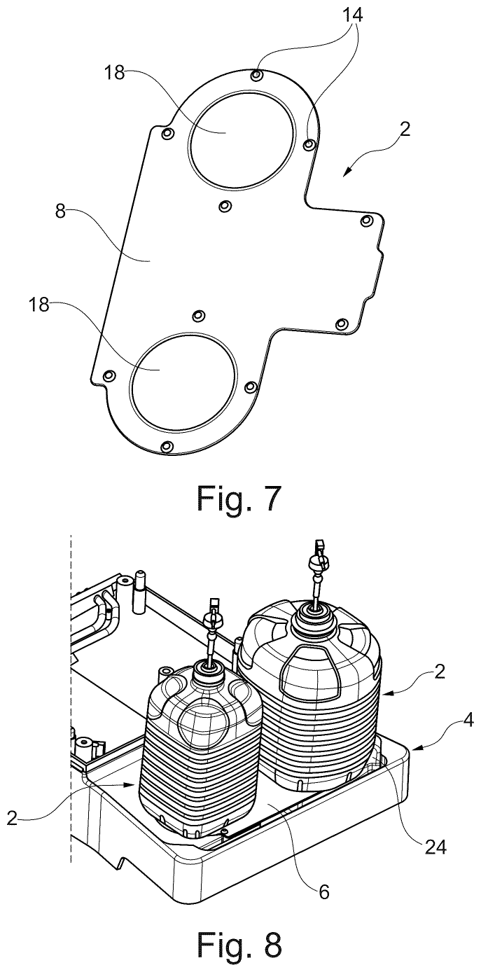

FIG. 7 shows the second surface 20 of the support plate 6. The openings 34 formed by the convex projection 22 on the second surface 20 are closed by the lids 36. The lids 36 can be welded to the support plate 36, in particular by laser welding. The second surface 20 also has a number of spacers 32, which protrude essentially vertically from the second surface 20. The spacers 32 are distributed around the circumference of the support plate 6.

FIG. 8 shows the two fluid containers 2 mounted on the support plate 6. The fluid containers 2 are each designed as bottles 46 and have a notch 26. This is why the support plate 6 is aligned in the first insertion orientation. This means that the two convex projections 22 point upward toward the bottles 46 and engage with the bottles 46. Since the edge 24 of the base 4 protrudes beyond the support plate 6, the edge 24 can provide additional support for the bottles 46. The two bottles 46 have different volumes. As long as the notches 26 of the bottles 46 fit onto the projections 22, the bottles 46 can be fastened by the projections 22.

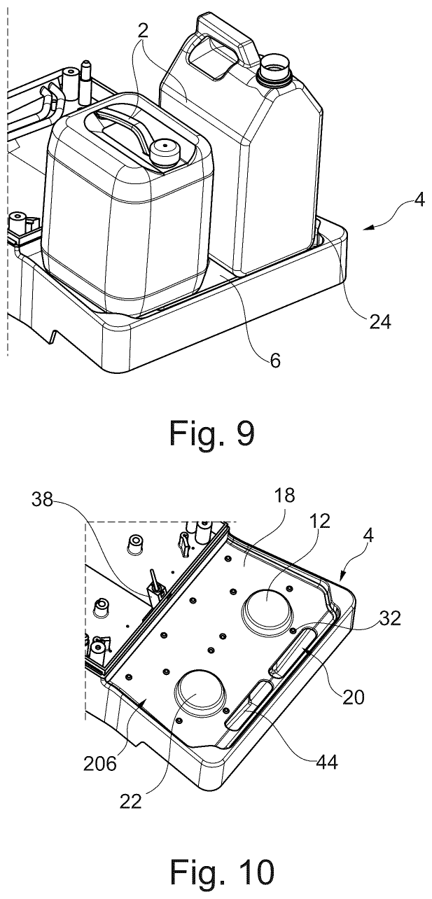

FIG. 9 shows two further fluid containers 2 which are positioned on the support plate 6 in the second insertion orientation. The fluid containers are shaped like canisters 48. The canisters 48 each have a flat floor section 50 from which four opposite side walls 52 protrude. The flat floor section 50 can be placed on the second surface 20 of the support plate 6. Thanks to the flat floor section 50, the canisters 48 have such a large support surface that no additional fastening element is necessary. The fluid containers 2 are supported at least in sections on the protruding edge 24 of the base 4.

FIG. 10 shows a support plate 206 according to a second embodiment. The differences between the support plates of the individual embodiments are described below. Otherwise, the support plate 206 according to the second embodiment has all the features of the support plate 6 according to the first embodiment. The support plate 206 is approximately rectangular in shape. The support plate 206 according to the second embodiment is larger than the support plate 6 shown in FIGS. 2 through 8. The support plate 206 covers the support section 8 of the base 4 and is enclosed by the protruding edge 24 of the base 4. The outer contour of the support plate is therefore adapted to the inner contour of the edge 24. The edge 24 protrudes beyond the support plate 206. This prevents fluid from leaking out of base 4. In the circumferential direction, the gap 16 is formed between the support plate 206 and the edge 24. The first surface 18 of the support plate 206 has the two convex projections 22. The support plate 206 also has the handling openings 44 and spacers 32.

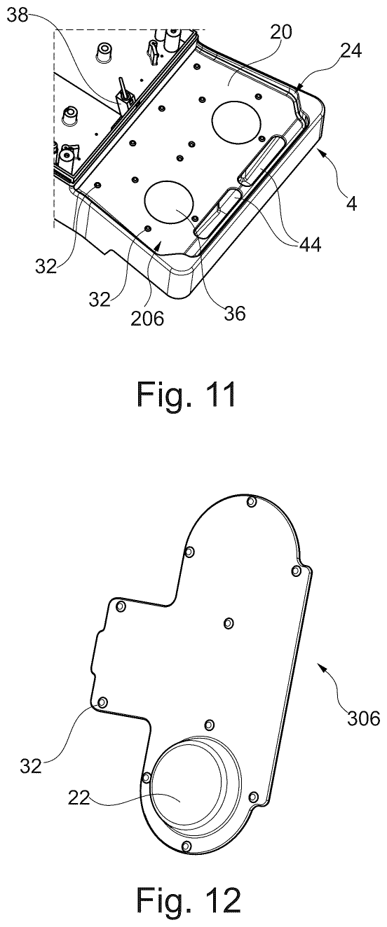

FIG. 11 shows the support plate 206 in the second insertion orientation. The openings 34 of the second surface 20 are closed by the lids 36. Thus, the second surface 20 is essentially flat/level. The second surface 20 also has the spacers 32. This means that the spacer 32 is spaced apart from the support plate 206 of the support section 8 of the base 4, and liquid can flow from the support plate 206 into the recess 10 of the base 4.

Of course, the support plate 6, 206 can also have two projections 22, each of which has different sizes and is therefore adapted for notches 26 with different sizes.

FIG. 12 shows a support plate 306 according to a third embodiment. The support plate 306 according to the third embodiment essentially corresponds to the support plate 6 according to the first embodiment. The only difference is that the support plate 306 has a convex projection 22. This means that the first surface 18 of the support plate 306 has two different resting spaces next to each other, one resting space being designed for a bottle 46 with a notch 26 and the other resting space being designed for a canister 48 with a flat floor section 50.

Of course, the support plate 206 according to the second embodiment can also have only one convex projection 22 and thus also form two different resting spaces next to each other.

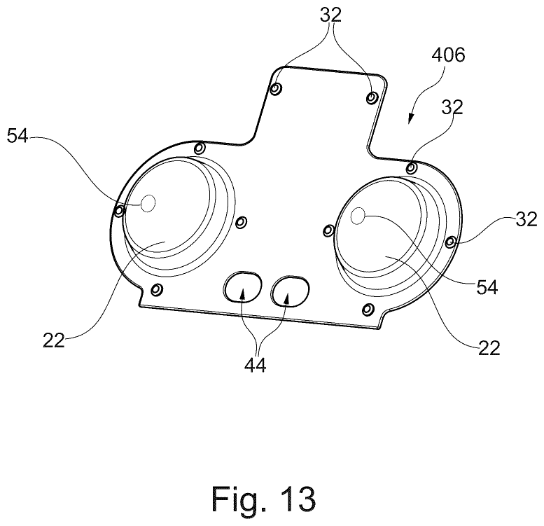

FIG. 13 shows a support plate 406 according to a fourth embodiment. The support plate 406 has a bore/fluid outlet bore 54 in each of the convex projections 22. The support plate 406 preferably has no lids 36 on the openings 34. This means that fluid can collect in the (deep) openings 34 in the second insertion orientation. The fluid can flow from the support plate 406 into the recess 10 through the fluid outlet bores 54. In further embodiments, the support plate 406 may have handling openings 44 for handling the support plate 406 and/or a number of protruding spacers 32.

### LIST OF REFERENCE SIGNS

1 dialysis machine
2 fluid container
4 base
6, 206, 306; 406 support plate
8 support section
10 recess
12 floor
14 support surface
16 gap
18 first surface
20 second surface
22 convex projection
24 edge
26 notch
28 outer surface of the projection
30 inner surface of the notch
32 spacers
34 opening
36 lid
38 leakage sensor
44 handling opening
46 bottle
48 canisters
50 floor section
52 side wall
54 fluid outlet bore

1. A dialysis machine comprising a removable support plate for a fluid container,
the removable support plate having a first surface and a second surface opposite the first surface,
the second surface differing from the first surface,
the first surface having at least one projection,
the at least one projection configured to engage in or be accommodated in a corresponding notch of the fluid container, and
the removable support plate being insertable into a receptacle section of the dialysis machine in a first insertion orientation or a second insertion orientation depending on a shape of a floor section of the fluid container, such that:
when the removable support plate is in the first insertion orientation, the first surface faces toward the receptacle section, and
when the removable support plate is in the second insertion orientation, the second surface faces toward the receptacle section.

2. The dialysis machine according to claim 1, wherein the receptacle section has a recess into which the at least one projection extends when the removable support plate is inserted in the first insertion orientation.

3. The dialysis machine according to claim 2, further comprising a leakage sensor that is attached to the receptacle section.

4. The dialysis machine according to claim 3, wherein the leakage sensor is attached at a lowest point of a floor of the recess.

5. The dialysis machine according to claim 4, wherein the floor of the recess is inclined relative to horizontal.

6. The dialysis machine according to claim 2, wherein a support surface is formed in the recess, on which the removable support plate rests.

7. The dialysis machine according to claim 6, wherein a gap is formed between the support surface and the removable support plate.

8. The dialysis machine according to claim 7, wherein the removable support plate has protruding spacers which form a clearance between the removable support plate and the support surface and form the gap.

9. The dialysis machine according to claim 7, wherein the support surface has protruding spacers which form a clearance between the removable support plate and the support surface and form the gap.

10. The dialysis machine according to claim 1, wherein the at least one projection forms an opening on the second surface.

11. The dialysis machine according to claim 10, wherein the opening is closed with a lid.

12. The dialysis machine according to claim 10, wherein the at least one projection has a bore.

13. The dialysis machine according to claim 12, wherein the bore is positioned at a point of the at least one projection that is farthest away from the first surface.

14. The dialysis machine according to claim 1, wherein the removable support plate is inclined relative to horizontal.

15. The dialysis machine according to claim 14, wherein the removable support plate is mounted on the receptacle section of the dialysis machine.

16. The dialysis machine according to claim 1, wherein the removable support plate is offset relative to an edge of the receptacle section so that the edge protrudes beyond the removable support plate.

17. A system comprising:
the dialysis machine according to claim 1; and
a fluid container,
the fluid container comprising a floor section and a notch in the floor section,
the at least one projection of the first surface engaging in or being accommodated in the notch.

18. A support plate for a dialysis machine for holding a fluid container, the support plate being removable and comprising:
a first surface; and
a second surface opposite the first surface that differs from the first surface;
the first surface having at least one projection,
the at least one projection configured to engage in or be accommodated in a corresponding notch of the fluid container, and
the support plate being inserted or insertable into a receptacle section of the dialysis machine in a first insertion orientation or a second insertion orientation depending on a shape of a floor section of the fluid container, such that:
when the support plate is in the first insertion orientation, the first surface faces toward the receptacle section, and
when the support plate is in the second insertion orientation, the second surface faces toward the receptacle section.
